(19) United States
(12) Patent Application Publication
(10) Pub. No.: US 2013/0157873 A1
(43) Pub. Date: Jun. 20, 2013

(54) METHODS OF ASSESSING A RISK OF DEVELOPING NECROTIZING MENINGOENCEPHALITIS

(71) Applicants: Matthew Huentelman, Phoenix, AZ (US); Scott Schatzberg, Phoenix, AZ (US); Renee Barber, Phoenix, AZ (US)

(72) Inventors: Matthew Huentelman, Phoenix, AZ (US); Scott Schatzberg, Phoenix, AZ (US); Renee Barber, Phoenix, AZ (US)

(73) Assignee: TRANSLATIONAL GENOMICS RESEARCH INSTITUTE, Phoenix, AZ (US)

(21) Appl. No.: 13/698,907

(22) PCT Filed: May 19, 2011
(86) PCT No.: PCT/US11/37262

§ 371 Date: Mar. 8, 2013

Related U.S. Application Data
(60) Provisional application No. 61/346,309, filed on May 19, 2010.

(51) Int. Cl. C12Q 1/68

(52) U.S. Cl. CPC C12Q 1/6883
USPC 506/2; 536/24.31; 536/24.33; 435/6.11; 435/6.12

(57) ABSTRACT

Methods of using single nucleotide polymorphisms (SNPs), SNP haplotype block, and haplotype to predict whether or not a subject will develop necrotizing meningoencephalitis (NME) and probe sets that facilitate those methods are disclosed. In particular, the subject is a canine species.

### BACKGROUND OF THE INVENTION

Necrotizing meningoencephalitis (NME) is a non-suppurative inflammatory disorder of the canine central nervous system. Overrepresented in Pug dogs, NME also occurs in other small breeds including the Maltese and Chihuahua. The etiology of NME is unknown but non-Mendelian inheritance has been demonstrated in Pug dogs, suggesting a role for genetic risk factors in the development of disease.

Necrotizing meningoencephalitis (NME) is an idiopathic inflammatory disorder of the central nervous system (CNS) that primarily affects young to middle aged toy breed dogs. NME has known non-Mendelian inheritance that shares clinical similarities with atypical variants of multiple sclerosis in humans. Inflammation in NME is characterized by mixed mononuclear cell infiltrates within the cerebral hemispheres and cortical leptomeninges with common clinical signs including seizures, depression, behavior change, circling and visual deficits. Similar to severe non-prototypical forms of multiple sclerosis (MS) such as Marburg variant, NME is overrepresented in females, is rapidly progressive and often carries a grave prognosis despite aggressive immunosuppressive treatment.

NME initially was identified in Pug dogs in the late 1960s and is known to have a strong familial association in this breed. Purebred dog populations provide a unique opportunity for mapping genetic traits and recent technological developments have made it possible to leverage dogs as a model for the study of human genetic disease. Dogs and humans share similar physiology with over half of the known canine diseases having a similar phenotype to analogous human diseases. An evaluation of canine NME, a disorder having clinical similarities to atypical, fulminant variants of MS in humans, is needed for identifying at risk and affected dogs, allowing development of targeted therapy and identifying similar genetic factors that are associated with the development of rapidly progressive MS in people.

Whether it is a human population or a canine population, the standard for measuring genetic variation among individuals in a population is the haplotype, which is the ordered combination of polymorphisms in the sequence of each form of a gene that exists in the population. Because haplotypes represent the variation across each form of a gene, they provide a more accurate and reliable measurement of genetic variation than individual polymorphisms. For example, while specific variations in gene sequences have been associated with a particular phenotype such as disease susceptibility (Roses A D supra; Ulbrecht M et al. 2000 Am J Respir Crit. Care Med 161: 469-74) and drug response (Wolfe C R et al. 2000 BMJ 320:987-90; Dahl B S 1997 Acta Psychiatr Scand 96 (Suppl 391): 14-21), in many other cases an individual polymorphism may be found in a variety of genomic backgrounds, i.e., different haplotypes, and therefore shows no definitive coupling between the polymorphism and the causative site for the phenotype (Clark A G et al. 1998 Am J Hum Genet. 63:595-612; Ulbrecht M et al. 2000 supra; Drysdale et al. 2000 PNAS 97:10483-10488). Thus, there is an unmet need for information on what haplotypes exist in the dog population that are associated with NME. Since canine NME is a disorder having clinical similarities to MS in humans, canine NME haplotype information would be useful in improving the efficiency and output of NME and MS diagnosis, prognosis, and the several steps in the drug discovery and development process, including target validation, identifying lead compounds, and early phase clinical trials of drugs for MS.

### BRIEF SUMMARY OF THE INVENTION

The present invention provides among other things:

It is an object of the invention to provide a test that classifies a subject into an NME disease risk group.

It is an object of the invention to provide a test that predicts whether or not a subject will develop NME.

The above and other objects may be achieved using methods involving receiving a sample from a subject, isolating nucleic acid from the sample, detecting one or more of the markers listed in Table 2 and classifying the subject into a cohort based upon the presence or absence of the marker. The marker may be directed directly such as by Sanger sequencing, pyrosequencing, SOLID sequencing, massively parallel sequencing, barcoded DNA sequencing, PCR, real-time PCR, quantitative PCR, microarray analysis of genomic DNA, restriction fragment length polymorphism analysis, allele specific ligation, and comparative genomic hybridization. Alternatively, the marker may be directed indirectly such as by microarray analysis of RNA, RNA in situ hybridization, RNAse protection assay, Northern blot, reverse transcription PCR, quantitative PCR, quantitative reverse transcription PCR, quantitative real-time reverse transcription PCR, reverse transcriptase treatment followed by direct sequencing, flow cytometry, immunohistochemistry, ELISA, Western Blot, immunoaffinity chromatography, HPLC, mass spectrometry, protein microarray analysis, PAGE analysis, isoelectric focusing, and 2D gel electrophoresis. The marker may be a marker that is associated with a high risk of developing NME and the subject is classified into a risk group comprising subjects with a high risk of developing NME if the marker is detected in the sample. The subject may be any animal including a dog such as a Pug Dog, Chihuahua, Maltese Terrier, West Highland White Terrier, Yorkshire Terrier, French Bulldog, and Pekingese.

The above and other objects may be achieved through the use of kits comprising a first probe capable of detecting a first SNP selected from the group listed in Table 2, a second probe capable of detecting a second SNP selected from Table 2; and wherein the probes are associated with a microarray of 1000 or fewer elements. The first probe may be capable of detecting a SNP associated with a higher risk of developing NME, or the second probe may be capable of detecting a SNP associated with a lower risk of developing NME.

### BRIEF DESCRIPTION OF THE FIGURES

FIG. 1 depicts the SNPs in DLA class II region on Chromosome 12, with alternative base as listed for A_{R}, the allele associated with NME risk, and A_{NR}, the non-risk allele.

FIG. 2 depicts the SNP in STYX region on Chromosome 8, with alternative base as listed for A_{R}, the allele associated with NME risk, and A_{NR}, the non-risk allele.

FIG. 3 depicts the genome-wide association results for 28 NME cases and 45 controls. (a) Fisher's exact tests were performed to compare SNP allele frequencies and negative log P values were plotted across the genome. The horizontal dotted line represents the threshold for significant association after Bonferroni correction of −log(P)>6.24 with a strong peak on chromosome 12 maintaining genome-wide significance. (b) MaxT 100,000 permutation testing was performed and negative log P values were plotted across the genome. The horizontal dotted line represents the threshold for significant association after permutation testing of −log(P)>1.3 with one SNP on chromosome 8 maintaining permuted significance.

FIG. 4 depicts haplotype analysis of NME-associated DLA II locus on chromosome 12. A 4.1 Mb region located from positions 4,713,392 to 8,834,652 with 19 haplotype blocks generated in Haploview. Negative log P values from single SNP associations were derived from genome-wide analysis after removal of population outliers and individual SNPs were plotted as red circles. The horizontal dotted line represents the threshold for significant association after Bonferroni correction of −log(P)>6.24.

FIG. 5 depicts haplotype analysis of NME-associated locus on chromosome 8. A 488 kb region located from positions 31,736,206 to 32,225,068 is shown with 4 haplotype blocks generated in Haploview. Negative log P values from single SNP associations were derived from genome-wide analysis after removal of population outliers and individual SNPs were plotted as red circles. The horizontal dotted line represents the threshold for significant association after Bonferroni correction of −log(P)>6.24. The additional blue region within each haplotype block represents MaxT 100,000 permuted haplotypes.

### DETAILED DESCRIPTION OF THE INVENTION

The disclosure provides a method of assigning a subject to a necrotizing meningoencephalitis (NME) risk group in order to assess the likelihood of the subject being afflicted with the disease. This method can be employed to assess the risk at early stages of disease progression. The method includes providing a biological sample from the subject, detecting a marker in a biological sample, which can be a haplotype associated with NME and assigning the subject to the NME risk group based upon the presence or absence of the haplotype. The method involves directly or indirectly detecting the presence or absence of the marker. Multiple markers disclosed herein may be used in combination to improve the accuracy, including two or more, three or more, four or more, five or more, or ten or more of the markers may be used.

Detection of the disease also includes detection of the haplotype by any SNPs/markers within the haplotype, but also indirectly through SNPs/markers outside the haplotype and leveraging linkage disequilibrium to identify carriers of the haplotype. In addition to determining a patient's relative risk for NME, the diagnosis may include prescribing therapeutic regimens to treat, prevent or delay onset of NME.

A haplotype may be any combination of one or more closely linked alleles inherited as a unit with little genetic shuffling across generations. An allele includes any form of a particular nucleic acid that may be recognized as a form of the particular nucleic acid on account of its location, sequence polymorphism, epigenetic modification or any other characteristic that may identify it as being a form of the particular gene. Alleles include but need not be limited to forms of a gene that include point mutations, silent mutations, deletions, frameshift mutations, single nucleotide polymorphisms (SNPs), inversions, translocations, heterochromatic insertions, and differentially methylated sequences relative to a reference gene sequence, whether alone or in combination. The presence or absence of an allele may be detected through the use of any process through which a specific nucleic acid molecule may be detected, including direct and indirect methods of detecting the presence or absence of the specific nucleic acid. Different alleles may, but need not, result in detectable differences in gene expression or protein functions. An allele of a gene may or may not encode proteins or peptides. Different alleles may differ in expression level, pattern, temporal or spatial specificity, and expression regulation. In the case of encoded proteins, the protein from different alleles may or may not be functional. Further, the protein may be gain-of-function, loss- of-function, or with altered function. An allele may also be called a mutation or a mutant. An allele may be compared to another allele that may be termed a wild type form of an allele. In some cases, the wild type allele is more common than the mutant.

Different combinations of polymorphisms may also be called haplotypes. The pholymorphism may be a single nucleotide polymorphism. The genetic sequences of different individuals are remarkably similar. When the chromosomes of two humans are compared, their DNA sequences can be identical for hundreds of bases. But at about one in every 1000 to 1,200 bases, on average, the sequences will differ. As such, one individual might have an A at that location, while another individual has a G, or a person might have extra bases at a given location or a missing segment of DNA. Differences in individual bases are the most common type of genetic variation. These genetic differences are known as single nucleotide polymorphisms (SNPs) (supra). SNPs act as markers to locate genes in DNA. Given the relatively close spacing between these SNPs, SNPs are typically inherited in blocks.

The difference of a single genetic variance such as a SNP can delineate a distinct haplotype. A “SNP haplotype block” or “haplotype block” is a nucleic acid sequence containing a group of SNPs or polymorphisms that do not appear to recombine independently resulting in reduced genetic variability but are passed together from generation to generation in variable-length blocks. The combination of polymorphisms, haplotype patterns and haplotype blocks may be referred to as a “haplotype” or “haplotype structure” in a nucleic acid sequence of interest. For example, a haplotype can be a set of SNPs, alleles, or genetic markers on a single chromatid that are genetically linked and thus are likely to be inherited as a unit. “Linked”, “linkage”, or “allelic association” means the preferential association of a particular allele or genetic marker with a specific allele or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles. A marker in linkage disequilibrium can be particularly useful in detecting susceptibility to disease (or other phenotype) notwithstanding that the marker does not cause the disease. For example, a marker (X) that is not itself a causative element of a disease, but which is in linkage disequilibrium with a gene (including regulatory sequences) (Y) that is a causative element of a phenotype, can be used detected to indicate susceptibility to the disease in circumstances in which the gene Y may not have been identified or may not be readily detectable.

As described above, two or more alleles likely to be inherited as a unit may be termed a haplotype block. When one or more haplotype blocks are associated with a pheonotypic trait, the haplotype block serves as a genetic marker represented by a genetic locus comprising one or more linked genetic variations that would be inherited as a unit more frequently than not in an individual having the associated phenotypic trait. Therefore the haplotype block, an/or the haplotype may also be used to identify individuals from biological samples for traits of interest. The haplotype block may, in turn, be used to identify individual polymorphic sites, or candidate genes for developing therapeutics and diagnostics.

When a SNP haplotype block is identified by a SEQ ID NO, a set of at least two SNPs that are associated with an allele of a gene are grouped together in a linkage unit or block. In one embodiment of the present invention, the presence of SNPs detected in a given haplotype block disclosed herein in a dog is associated with a greater risk that the dog will develop NME, if it has not yet shown symptoms of NME; whereas the absence of SNPs detected in a given haplotype block disclosed herein in a dog is associated with a lesser risk that the dog will develop NME. A nucleic acid may be termed to be specific to a SNP haplotype block or specific to a SNP within a haplotype block. A nucleic acid specific to a haplotype block or a SNP within a haplotype block contains sequence that is complementary to one of the double stranded nucleic acid sequence of at least one SNP that is grouped within that haplotype block. Such nucleic acids may be complementary to a SNP that is associated with the nucleotide sequence in the linkage unit or block or any other SNP associated within the haplotype identified by the SNP haplotype block.

A marker may be any molecular structure produced by a cell, expressed inside the cell, accessible on the cell surface, or secreted by the cell. A marker may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination. A marker may also be called a target and the terms are used interchangeably.

A marker may be represented by the sequence of a nucleic acid from which it can be derived or any other chemical structure. Examples of such nucleic acids include miRNA, tRNA, siRNA, mRNA, cDNA, or genomic DNA sequences including complimentary sequences. Alternatively, a marker may be represented by a protein sequence. The concept of a marker is not limited to the products of the exact nucleic acid sequence or protein sequence by which it may be represented. Rather, a marker encompasses all molecules that may be detected by a method of assessing the expression of the marker.

Examples of molecules encompassed by a marker represented by a particular sequence or structure include point mutations, silent mutations, deletions, frameshift mutations, translocations, alternative splicing derivatives, differentially methylated sequences, differentially modified protein sequences, truncations, soluble forms of cell membrane associated markers, and any other variation that results in a product that may be identified as the marker. The following nonlimiting examples are included for the purposes of clarifying this concept: If expression of a specific marker in a sample is assessed by RTPCR, and if the sample expresses an mRNA sequence different from the sequence used to identify the specific marker by one or more nucleotides, but the marker may still be detected using RTPCR, then the specific marker encompasses the sequence present in the sample. Alternatively if expression of a specific marker in a sample is assessed by an antibody and the amino acid sequence of the marker in the sample differs from a sequence used to identify marker by one or more amino acids, but the antibody is still able to bind to the version of the marker in the sample, then the specific marker encompasses the sequence present in the sample.

In the present invention, the marker represented by a group of linked SNPs, “haplotype block,” or “haplotype” may be detected by a variety of methodologies or procedures that are well know in the art including, but not limited to, nucleic acid hybridization, antibody binding, activity assay, polymerase chain reaction (PCR), S1 nuclease assay and via gene chip or microarray as well as any other assay known in the art that may be used to detect the SNPs associated with a haplotype or the gene product produced from the gene of the haplotype including mRNA and protein. Hybridization of a SNP-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin interactions, salt bridges, hydrophobic interactions, chemical linkages, UV cross-linking baking, etc. SNP-specific oligonucleotides may be synthesized directly on the solid support or attached to the solid support subsequent to synthesis. Solid-supports suitable for use in detection methods of the disclosure include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the SNP-specific oligonucleotide or target nucleic acid. Detecting the nucleotide or nucleotide pair of interest may also be determined using a mismatch detection technique, including but not limited to the RNase protection method using riboprobes (Winter et al. (1985) Proc. Natl. Acad. Sci. USA 82:7575; Meyers et al. (1985) Science 230:1242) and proteins which recognize nucleotide mismatches, such as the E. coli mutS protein (Modrich (1991) Ann. Rev. Genet. 25:229-53). Alternatively, variant SNPs or variant alleles can be identified by single strand conformation polymorphism (SSCP) analysis (Orita et al. (1989) Genomics 5:874-9); Humphries et al. (1996) in MOLECULAR DIAGNOSIS OF GENETIC DISEASES, Elles, ed., pp. 321-340) or denaturing gradient gel electrophoresis (DGGE) (Wartell et al. (1990) Nucl. Acids Res. 18:2699706); Sheffield et al. (1989) Proc. Natl. Acad. Sci. USA 86:232-6).

A polymerase-mediated primer extension method may also be used to identify the polymorphism(s). Several such methods have been described in the patent and scientific literature and include the “Genetic Bit Analysis” method (WO 92/15712) and the ligase/polymerase mediated genetic bit analysis (U.S. Pat. No. 5,679,524. Related methods are disclosed in WO 91/102087, WO 90/09455, WO 95/17676, and U.S. Pat. Nos. 5,302,509 and 5,945,283. Extended primers containing the complement of the polymorphism may be detected by mass spectrometry as described in U.S. Pat. No. 5,605,798. Another primer extension method is allele-specific PCR (Ruano et al. (1989) Nucl. Acids Res. 17:8392; Ruano et al. (1991) Nucl. Acids Res. 19:6877-82); WO 93/22456; Turki et al. (1995) 1. Clin. Invest. 95:1635-41). The haplotype for a gene of an individual may also be determined by hybridization of a nucleic acid sample containing one or both copies of the gene, mRNA, cDNA or fragment(s) thereof, to nucleic acid arrays and sub-arrays such as described in WO 95/112995. The arrays would contain a battery of SNP-specific or allele specific oligonucleotides representing each of the polymorphic sites to be included in the haplotype.

Detecting the presence or absence of a marker disclosed herein or a close isoform thereof may be carried out either directly or indirectly by any suitable methodology. A variety of techniques are known to those skilled in the art (supra). All generally involve receiving a biological sample containing DNA or protein from the subject, and then detecting whether or not the marker or a close isoform thereof is present in the sample. and then determining the presence or absence of the marker in the sample.

The marker may be detected by any of a number of methods. Direct methods of detecting the presence of an allele include but are not limited to any form of DNA sequencing including Sanger, next generation sequencing, pyrosequencing, SOLID sequencing, massively parallel sequencing, pooled, and barcoded DNA sequencing or any other sequencing method now known or yet to be disclosed; PCR-based methods such as real-time PCR, quantitative PCR, reverse transcription PCR or any combination of these; allele specific ligation; comparative genomic hybridization; or any other method that allows the detection of a particular nucleic acid sequence within a sample or enables the differentiation of one nucleic acid from another nucleic acid that differs from the first nucleic acid by one or more nucleotides.

A nucleic acid-containing sample used for various marker detection methods may be from a subject suspected of having or being at risk of having NME. Nucleic acids may include but need not be limited to RNA, cDNA, tRNA, mitochondrial DNA, plasmid DNA, siRNA, genomic DNA, or any other naturally occurring or artificial nucleic acid molecule. The sample may be any type of sample derived from the subject, including any fluid or tissue that may contain one or more markers associated with the haplotype. Examples of sources of samples include but are not limited to biopsy or other in vivo or ex vivo analysis of prostate, breast, skin, muscle, fascia, brain, endometrium, lung, head and neck, pancreas, small intestine, blood, liver, testes, ovaries, colon, skin, stomach, esophagus, spleen, lymph node, bone marrow, kidney, placenta, or fetus. In some aspects of the invention, the sample comprises a fluid sample, such as peripheral blood, lymph fluid, ascites, serous fluid, pleural effusion, sputum, cerebrospinal fluid, amniotic fluid, lacrimal fluid, stool, or urine.

The subject may be any organism subject or susceptible to NME or MS including mammals, further including humans. The animal may be a canine such as a Pug Dog, Chihuahua, West Highland White Terrier, Pekingese, Labrador retriever, Golden retriever, Beagle, German shepherd, Dachshund, Yorkshire terrier, Boxer, Poodle, Shih tzu, Miniature schnauzer, Pomeranian, Cocker spaniel, Rottweiler, Bulldog, Shetland sheepdog, Boston terrier, Miniature pinscher, Maltese, German shorthaired pointer, Doberman pinscher, Siberian husky, Pembroke welsh corgi, Basset hound, Bichon frise, and other existing or non-existing breeds.

Examples of indirect methods of detection include any nucleic acid detection method including the following nonlimiting examples, microarray analysis, RNA in situ hybridization, RNAse protection assay, Northern blot, reverse transcriptase PCR, quantitative PCR, quantitative reverse transcriptase PCR, quantitative real-time reverse transcriptase PCR, reverse transcriptase treatment followed by direct sequencing, direct sequencing of genomic DNA, or any other method of detecting a specific nucleic acid now known or yet to be disclosed. Other examples include any process of assessing protein expression including flow cytometry, immunohistochemistry, ELISA, Western blot, and immunoaffinity chromatograpy, HPLC, mass spectrometry, protein microarray analysis, PAGE analysis, isoelectric focusing, 2-D gel electrophoresis, or any enzymatic assay.

In Sanger Sequencing, a single-stranded DNA template, a primer, a DNA polymerase, \nucleotides and a label such as a radioactive label conjugated with the nucleotide base or a fluorescent label conjugated to the primer, and one chain terminator base comprising a dideoxynucleotide (ddATP, ddGTP, ddCTP, or ddTTP, are added to each of four reaction (one reaction for each of the chain terminator bases). The sequence may be determined by electrophoresis of the resulting strands. In dye terminator sequencing, each of the chain termination bases is labeled with a fluorescent label of a different wavelength which allows the sequencing to be performed in a single reaction.

In pyrosequencing, the addition of a base to a single stranded template to be sequenced by a polymerase results in the release of a pyrophosphate upon nucleotide incorporation. An ATP sulfyrlase enzyme converts pyrophosphate into ATP which in turn catalyzes the conversion of luciferin to oxyluciferin which results in the generation of visible light that is then detected by a camera.

In SOLID sequencing, the molecule to be sequenced is fragmented and used to prepare a population of clonal magnetic beads (in which each bead is conjugated to a plurality of copies of a single fragment) with an adaptor sequence and alternatively a barcode sequence. The beads are bound to a glass surface. Sequencing is then performed through 2-base encoding.

In massively parallel sequencing, randomly fragmented targeted DNA is attached to a surface. The fragments are extended and bridge amplified to create a flow cell with clusters, each with a plurality of copies of a single fragment sequence. The templates are sequenced by synthesizing the fragments in parallel. Bases are indicated by the release of a fluorescent dye correlating to the addition of the particular base to the fragment.

Other methods used to assess expression include the use of natural or artificial ligands capable of specifically binding a marker. Such ligands include antibodies, antibody complexes, conjugates, natural ligands, small molecules, nanoparticles, or any other molecular entity capable of specific binding to a marker. Antibodies may be monoclonal, polyclonal, or any antibody fragment including an Fab, F(ab)2, Fv, scFv, phage display antibody, peptibody, multispecific ligand, or any other reagent with specific binding to a marker. Ligands may be associated with a label such as a radioactive isotope or chelate thereof, dye (fluorescent or nonfluorescent,) stain, enzyme, metal, or any other substance capable of aiding a machine or a human eye from differentiating a cell expressing a marker from a cell not expressing a marker. Additionally, expression may be assessed by monomeric or multimeric ligands associated with substances capable of killing the cell. Such substances include protein or small molecule toxins, cytokines, pro-apoptotic substances, pore forming substances, radioactive isotopes, or any other substance capable of killing a cell.

Other markers may also be used that are associated with the markers disclosed herein such as SNPs or other polymorphic markers that are in close enough proximity to have a statistically significant association with the marker disclosed herein (e.g., other markers in linkage disequilibrium with a marker disclosed herein). For example, if a marker or a close isoform thereof is detected in the subject, then the subject may be placed into a group either at higher or lower risk for NME depending on which marker or close isoform thereof is identified (i.e., a significant enough number of markers associated with a haplotype).

The disclosure also provides sets of molecular probes for detection, including at least two probes capable of detecting, directly or indirectly, a marker disclosed herein associated with increased or decreased risk of NME, wherein the molecular probes are not associated with a microarray of greater than 1000 elements, a microarray with greater than 500 elements, a microarray with greater than 100 elements, a microarray with greater than 50 elements, or are not associated with a microarray. Such sets of two or more probes may include at least one probe capable of detecting, directly or indirectly, a marker disclosed herein associated with higher risk of developing NME and at least one other probe is capable of detecting, directly or indirectly, a marker disclosed herein associated with lower risk of developing NME.

If a marker can be detected through expression level alteration, the expression of the marker in a sample may be compared to a level of expression predetermined to predict the presence or absence of a particular physiological characteristic. The level of expression may be derived from a single control or a set of controls. A control may be any sample with a previously determined level of expression. A control may comprise material within the sample or material from sources other than the sample. Alternatively, the expression of a marker in a sample may be compared to a control that has a level of expression predetermined to signal or not signal a cellular or physiological characteristic. This level of expression may be derived from a single source of material including the sample itself or from a set of sources. Comparison of the expression of the marker in the sample to a particular level of expression results in a prediction that the sample exhibits or does not exhibit the cellular or physiological characteristic.

Prediction of a cellular or physiological characteristic includes the prediction of any cellular or physiological state that may be predicted by assessing the expression of a marker. Examples include the identity of a cell as a particular cell including a particular normal or diseased cell type, the likelihood that one or more diseases is present or absent, the likelihood that a present disease will progress, remain unchanged, or regress, the likelihood that a disease will respond or not respond to a particular therapy, or any other disease outcome. Further examples include the likelihood that a cell will move, senesce, apoptose, differentiate, metastasize, or change from any state to any other state or maintain its current state.

One type of cellular or physiological characteristic is the risk that a particular disease outcome will occur. Assessing this risk includes the performing of any type of test, assay, examination, result, readout, or interpretation that correlates with an increased or decreased probability that an individual has had, currently has, or will develop a particular disease, disorder, symptom, syndrome, or any condition related to health or bodily state. Examples of disease outcomes include, but need not be limited to survival, death, progression of existing disease, remission of existing disease, initiation of onset of a disease in an otherwise disease-free subject, or the continued lack of disease in a subject in which there has been a remission of disease. Assessing the risk of a particular disease encompasses diagnosis in which the type of disease afflicting a subject is determined. Assessing the risk of a disease outcome also encompasses the concept of prognosis. A prognosis may be any assessment of the risk of disease outcome in an individual in which a particular disease has been diagnosed. Assessing the risk further encompasses prediction of therapeutic response in which a treatment regimen is chosen based on the assessment. Assessing the risk also encompasses a prediction of overall survival after diagnosis.

Determining whether or not the presence of an allele signifies a physiological or cellular characteristic may be assessed by any of a number of methods. The skilled artisan will understand that numerous methods may be used to select a marker or a plurality of markers that signifies a particular physiological or cellular characteristic. In diagnosing the presence of a disease, a threshold value may be obtained by performing the assay method on samples obtained from a population of patients having a certain type of disease (NME for example,) and from a second population of subjects that do not have the disease. In assessing disease outcome or the effect of treatment, a population of patients, all of which may develop a disease such as NME, may be followed for a period of time. After the period of time expires, the population may be divided into two or more groups. For example, the population may be divided into a first group of patients who did develop NME and a second group of patients who did not develop NME. Examples of endpoints include occurrence of one or more symptoms of disease, death or other states to which the given disease may progress. If presence of the marker in a sample statistically aligns with one group relative to the other group, the subject from which the sample was derived may be assigned a risk of having the same outcome as the patient group that differentially displays the marker.

Other methods may be used to assess how accurately the presence or absence of a marker signifies a particular physiological or cellular characteristic. Such methods include a positive likelihood ratio, negative likelihood ratio, odds ratio, and/or hazard ratio. In the case of a likelihood ratio, the likelihood that the presence or absence of the marker would be found in a sample with a particular cellular or physiological characteristic is compared with the likelihood that the presence or absence of the marker would be found in a sample lacking the particular cellular or physiological characteristic.

An odds ratio measures effect size and describes the amount of association or non-independence between two groups. An odds ratio is the ratio of the odds of a marker being present or absent in one set of samples versus the odds of the marker being present or absent in the other set of samples. An odds ratio of 1 indicates that the event or condition is equally likely to occur in both groups. An odds ratio grater or less than 1 indicates that presence or absence of the marker is more likely to occur in one group or the other depending on how the odds ratio calculation was set up.

A hazard ratio may be calculated by estimate of relative risk. Relative risk is the chance that a particular event will take place. It is a ratio of the probability that an event such as development or progression of a disease will occur in samples in which a particular marker is present over the probability that the event will occur in samples in which the particular marker is absent. Alternatively, a hazard ratio may be calculated by the limit of the number of events per unit time divided by the number at risk as the time interval decreases. In the case of a hazard ratio, a value of 1 indicates that the relative risk is equal in both the first and second groups; a value greater or less than 1 indicates that the risk is greater in one group or another, depending on the inputs into the calculation.

### EXAMPLE

The purpose of this investigation was to identify disease susceptibility loci for NME through genome-wide association (GWA) of single nucleotide polymorphisms (SNPs) in affected and non-affected Pug dogs. 170,000 SNPs, genome-wide association was performed on a small number of case and control dogs to determine disease susceptibility loci in canine necrotizing meningoencephalitis (NME), a disorder with known non-Mendelian inheritance that shares clinical similarities with atypical variants of multiple sclerosis in humans. Genotyping of 30 NME-affected Pug dogs and 68 healthy, control Pugs identified two loci associated with NME, including a region within dog leukocyte antigen class II on chromosome 12 that remained significant after Bonferroni correction. Our results support the utility of this high density SNP array, confirm that dogs are a powerful model for mapping complex genetic disorders and provide important preliminary data to support in depth genetic analysis of NME in numerous affected breeds

### Study Population

Purebred Pug dogs were used for the case-control genome-wide association study. Cases were verified to have NME based on signalment, clinical history and independent evaluation of hematoxylin and eosin brain sections by a veterinary neuropathologist. Cases ranged in age from 4 to 84 months (mean=18 months, median=26 months) and consisted of 11 males and 19 females. Control dogs had no evidence of neurological or autoimmune disease, ranged in age from 5 to 204 months (mean=60 months, median=48 months) at the time of sample collection and consisted of 30 males and 38 females. Control dogs were followed for 18 months after sample collection to verify that they did not develop neurological or autoimmune disease.

### SNP Genotyping

Genomic DNA was isolated using the Qiagen (Valencia, Calif.) Gentra Puregene Tissue Kit or Qiagen DNeasy Blood and Tissue Kit. SNP genotyping was performed with the Illumina (San Diego, Calif.) CanineHD Genotyping BeadChip using the Illumina BeadArray reader following the manufacturer's protocol. Genomic DNA was isolated from 30 Pug dogs with histopathologically confirmed NME and 68 healthy, control Pug dogs without evidence of neurological or autoimmune disease. Genomic DNA quality was assessed with 2% agarose gel electrophoresis and quantity with fluorometric dsDNA quantification. Genome-wide association of >100,000 SNPs was performed using the Illumina Canine Infinium® HD BeadChip, and SNPs were analyzed with PLINK (http://pngu.mgh.harvard.edu/purcell/plink/) with a minor allele frequency of >5% and call rate of >98%.

### Statistical Analysis

Genotyping was performed on 98 dogs, including 30 NME cases and 68 controls. Genome-wide analysis was performed with PLINK (Purcell et al. 2007). Concordance on duplicate samples was 99.96%. Only samples with a call rate of >95% were included, resulting in analysis of 28 NME cases and 66 controls. A total of 172,115 SNPs were genotyped. Classic multidimensional scaling (Purcell et al. 2007) using a call rate of >97% and MAF of >0.10 was performed on 85,366 SNPs to determine population stratification, and 21 controls that were not clustered with the main population of dogs were excluded resulting in a final population of 28 NME cases and 45 controls for analysis. These 45 control dogs ranged in age from 5 to 204 months (mean=80 months, median=48 months). Prior to analysis, 7,324 SNPs were excluded for failure to reach the call rate threshold (>95%) and 81,001 SNPs were excluded for failure to reach the MAF threshold (>0.05). In total, 86,692 SNPs were used for analysis. Bonferroni correction was applied to account for multiple hypothesis testing with a resulting P value of 5.77×10⁻⁷ across 86,692 SNPs for genome-wide significance. To further evaluate genome-wide significance, MaxT permutation testing (Purcell et al. 2007) of 100 000 permutations was applied.

### Results

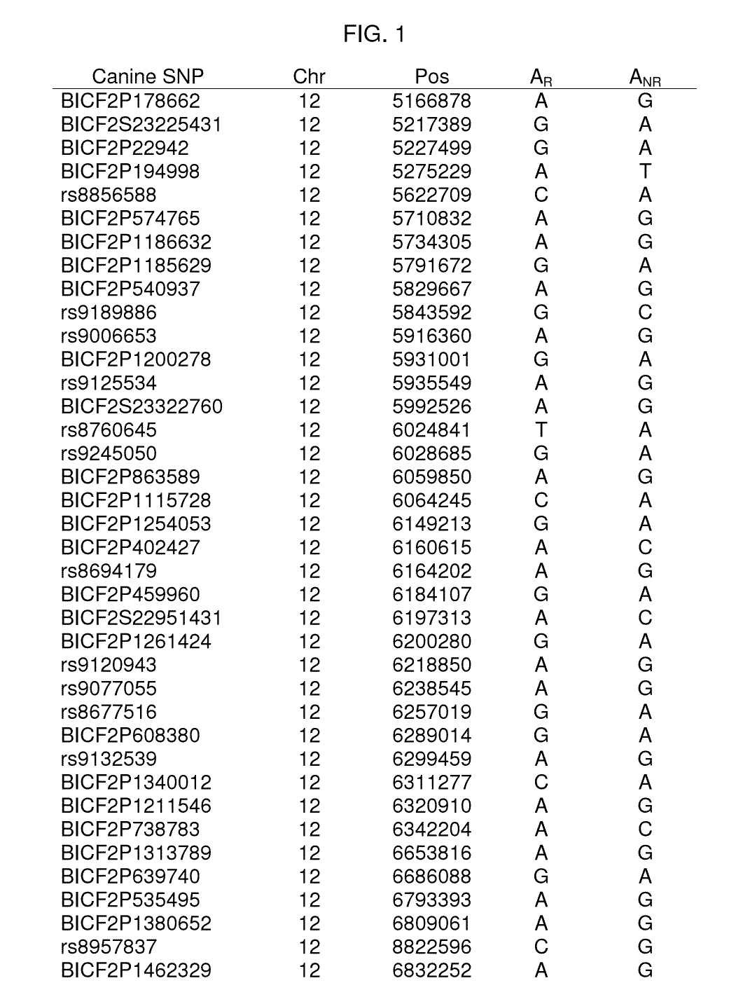

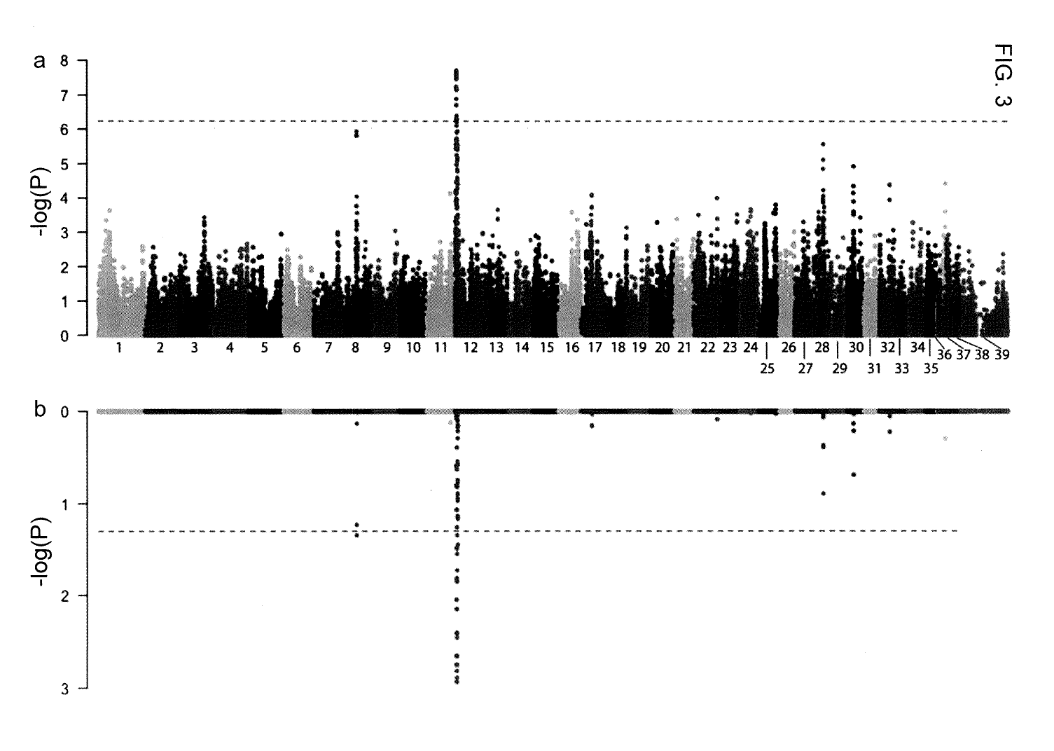

Initial genotyping was performed on 30 NME cases and 68 controls across 172,115 SNPs. After quality filtering and exclusion of population outliers, analysis of 28 NME cases and 45 controls across 86,692 SNPs identified two disease-associated loci that reached genome-wide significance with correction for multiple hypothesis testing. The strongest association was on chromosome 12 where 35 SNPs within the DLA class II region reached genome-wide significance after Bonferroni correction (raw P value for Bonferroni genome-wide significance is p<5.77×10⁻⁷) with the highest SNP having an odds ratio of 16.1 (95% CI: 4.7-55.5) (FIG. 3A and Table 1). FIG. 1 showed the genome-wide association results for 28 NME cases and 45 controls. In FIG. 1A, Fisher's exact tests were performed to compare SNP allele frequencies and negative log P values were plotted across the genome. The horizontal dotted line represents the threshold for significant association after Bonferroni correction of −log(P)>6.24 with a strong peak on chromosome 12 maintaining genome-wide significance.

TABLE 1
SNPs with genome-wide significance after Bonferroni correction

| Canine SNP | Chr | Pos | A_{R}/A_{NR} | F_{A}/F_{U} | P_{raw} | P_{genome} | OR (95% CI) | | Gene |
|---|---|---|---|---|---|---|---|---|---|
| CF2P178662 | 12 | 5166878 | A/G | 0.95/0.52 | 2.36 × 10⁻⁸ | 0.0020 | 16.1 | (4.7-55.5) | RT1-Db2 |
| BICF2S23225431 | 12 | 5217389 | G/A | 0.86/0.36 | 2.87 × 10⁻⁹ | 0.0002 | 10.5 | (4.4-25) | RT1-Db2 |
| BICF2P22942 | 12 | 5227499 | G/A | 0.86/0.36 | 2.87 × 10⁻⁹ | 0.0002 | 10.5 | (4.4-25) | RT1-Db2 |
| BICF2P194998 | 12 | 5275229 | A/T | 0.79/0.30 | 8.59 × 10⁻⁹ | 0.0007 | 8.7 | (4-19.2) | |
| rs8856588 | 12 | 5622709 | C/A | 0.79/0.30 | 8.59 × 10⁻⁹ | 0.0007 | 8.7 | (4-19.2) | COL11A2 |
| BICF2P574765 | 12 | 5710832 | A/G | 0.79/0.30 | 8.59 × 10⁻⁹ | 0.0007 | 8.7 | (4-19.2) | bing4-a |
| BICF2P1186632 | 12 | 5734305 | A/G | 0.79/0.29 | 7.24 × 10⁻⁹ | 0.0006 | 8.9 | (4.1-19.7) | TAPBP |
| BICF2P1185629 | 12 | 5791672 | G/A | 0.79/0.30 | 8.59 × 10⁻⁹ | 0.0007 | 8.7 | (4-19.2) | KIFC1 |
| BICF2P540937 | 12 | 5829667 | A/G | 0.79/0.32 | 6.27 × 10⁻⁸ | 0.0054 | 7.9 | (3.6-17.1) | CUTA |
| rs9189886 | 12 | 5843592 | G/C | 0.93/0.51 | 5.95 × 10⁻⁸ | 0.0052 | 12.4 | (4.1-37.3) | Syngap1 |
| rs9006653 | 12 | 5916360 | A/G | 0.79/0.33 | 8.07 × 10⁻⁸ | 0.0070 | 7.5 | (3.4-16.2) | Ppdpfb |
| BICF2P1200278 | 12 | 5931001 | G/A | 0.79/0.29 | 7.24 × 10⁻⁹ | 0.0006 | 8.9 | (4.1-19.7) | Ppdpfb |
| rs9125534 | 12 | 5935549 | A/G | 0.79/0.30 | 8.59 × 10⁻⁹ | 0.0007 | 8.7 | (4-19.2) | Ppdpfb |
| BICF2S23322760 | 12 | 5992526 | A/G | 0.79/0.30 | 8.59 × 10⁻⁹ | 0.0007 | 8.7 | (4-19.2) | Ppdpfb |
| rs8760645 | 12 | 6024841 | T/A | 0.79/0.30 | 8.59 × 10⁻⁹ | 0.0007 | 8.7 | (4-19.2) | Ppdpfb |
| rs9245050 | 12 | 6028685 | G/A | 0.79/0.30 | 8.59 × 10⁻⁹ | 0.0007 | 8.7 | (4-19.2) | Ppdpfb |
| BICF2P863589 | 12 | 6059850 | A/G | 0.88/0.47 | 4.83 × 10⁻⁷ | 0.0419 | 8 | (3.3-19.7) | Ppdpfb |
| BICF2P1115728 | 12 | 6064245 | C/A | 0.88/0.47 | 4.83 × 10⁻⁷ | 0.0419 | 8 | (3.3-19.7) | Ppdpfb |
| BICF2P1254053 | 12 | 6149213 | G/A | 0.84/0.35 | 7.23 × 10⁻⁹ | 0.0006 | 9.6 | (4.2-22.2) | MLN |
| BICF2P402427 | 12 | 6160615 | A/C | 0.79/0.30 | 8.59 × 10⁻⁹ | 0.0007 | 8.7 | (4-19.2) | Ggnbp1 |
| rs8694179 | 12 | 6164202 | A/G | 0.84/0.35 | 7.23 × 10⁻⁹ | 0.0006 | 9.6 | (4.2-22.2) | Ggnbp1 |
| BICF2P459960 | 12 | 6184107 | G/A | 0.84/0.35 | 7.23 × 10⁻⁹ | 0.0006 | 9.6 | (4.2-22.2) | Ggnbp1 |
| BICF2S22951431 | 12 | 6197313 | A/C | 0.79/0.30 | 8.59 × 10⁻⁹ | 0.0007 | 8.7 | (4-19.2) | Ggnbp1 |
| BICF2P1261424 | 12 | 6200280 | G/A | 0.84/0.35 | 7.23 × 10⁻⁹ | 0.0006 | 9.6 | (4.2-22.2) | Ggnbp1 |
| rs9120943 | 12 | 6218850 | A/G | 0.84/0.35 | 7.23 × 10⁻⁹ | 0.0006 | 9.6 | (4.2-22.2) | |
| rs9077055 | 12 | 6238545 | A/G | 0.84/0.35 | 7.23 × 10⁻⁹ | 0.0006 | 9.6 | (4.2-22.2) | |
| rs8677516 | 12 | 6257019 | G/A | 0.86/0.36 | 2.87 × 10⁻⁹ | 0.0002 | 10.5 | (4.4-25) | |
| BICF2P608380 | 12 | 6289014 | G/A | 0.86/0.36 | 2.87 × 10⁻⁹ | 0.0002 | 10.5 | (4.4-25) | |
| rs9132539 | 12 | 6299459 | A/G | 0.79/0.30 | 1.96 × 10⁻⁸ | 0.0017 | 8.5 | (3.9-18.6) | |
| BICF2P1340012 | 12 | 6311277 | C/A | 0.79/0.30 | 8.59 × 10⁻⁹ | 0.0007 | 8.7 | (4-19.2) | |
| BICF2P1211546 | 12 | 6320910 | A/G | 0.79/0.30 | 8.59 × 10⁻⁹ | 0.0007 | 8.7 | (4-19.2) | |
| BICF2P738783 | 12 | 6342204 | A/C | 0.79/0.29 | 6.06 × 10⁻⁹ | 0.0005 | 9.2 | (4.1-20.3) | LOC1127664 |
| BICF2P1313789 | 12 | 6653816 | A/G | 0.79/0.34 | 1.89 × 10⁻⁹ | 0.0163 | 7.1 | (3.3-15.4) | NUDT3 |
| BICF2P1380652 | 12 | 6809061 | A/G | 0.79/0.33 | 8.07 × 10⁻⁸ | 0.0070 | 7.5 | (3.4-16.2) | SPDEF |
| BICF2P1462329 | 12 | 6832252 | A/G | 0.79/0.33 | 8.07 × 10⁻⁸ | 0.0070 | 7.5 | (3.4-16.2) | SPDEF |

In Table 1, Chr stands for chromosome; Pos stands for physical position; A_{R}, stands for risk allele; A_{NR} stands for nonrisk allele; F_{A} stands for allele frequency in cases; F_{U} stands for allele frequency in controls; and OR stands for odds ratio. (Same below)

Permutation testing using 100,000 permutations identified an additional four SNPs that reached genome-wide permuted significance within the DLA II locus and a second region of significance within the STYX gene on chromosome 8 (P_{raw}=2.11×10⁻⁶, P_{permuted}=0.045) with an odds ratio of 5.9 (95% CI: 2.7-12.5) (FIG. 3B and Table 2). FIG. 1B showed that MaxT 100,000 permutation testing was performed and negative log P values were plotted across the genome. The horizontal dotted line represents the threshold for significant association after permutation testing of −log(P)>1.3 with one SNP on chromosome 8 maintaining permuted significance.

TABLE 2
New SNPs discovered with genome-wide significance after permutation testing

| Canine SNP | Chr | Pos | A_{R}/A_{NR} | F_{A}/F_{U} | P_{raw} | P_{permuted} | OR [95% CI] | Gene |
|---|---|---|---|---|---|---|---|---|
| BICF2S23516667 | 8 | 31971609 | A/G | 0.73/0.32 | 2.11 × 10⁻⁶ | 0.0452 | 5.9 [2.8-12.5] | STYX |
| BICF2P639740 | 12 | 6686088 | G/A | 0.79/0.33 | 1.30 × 10⁻⁷ | 0.0283 | 7.5 [3.4-16.4] | NUDT3 |
| BICF2P535495 | 12 | 6793393 | A/G | 0.79/0.33 | 1.30 × 10⁻⁷ | 0.0453 | 7.5 [3.4-16.4] | MGC8455 |
| rs8957837 | 12 | 8822596 | C/G | 0.77/0.33 | 3.94 × 10⁻⁷ | 0.0356 | 6.7 [3.1-14.6] | LOC112577 |

To account for the fact that several of the control dogs were younger than the mean age of disease onset at the time of sample acquisition, the data was re-analyzed excluding all control dogs less than 24 months of age. Both the DLA and chromosome 8 regions remained significant with Bonferroni correction and permutation testing, respectively, but the significance was not improved by this exclusion (data not shown).

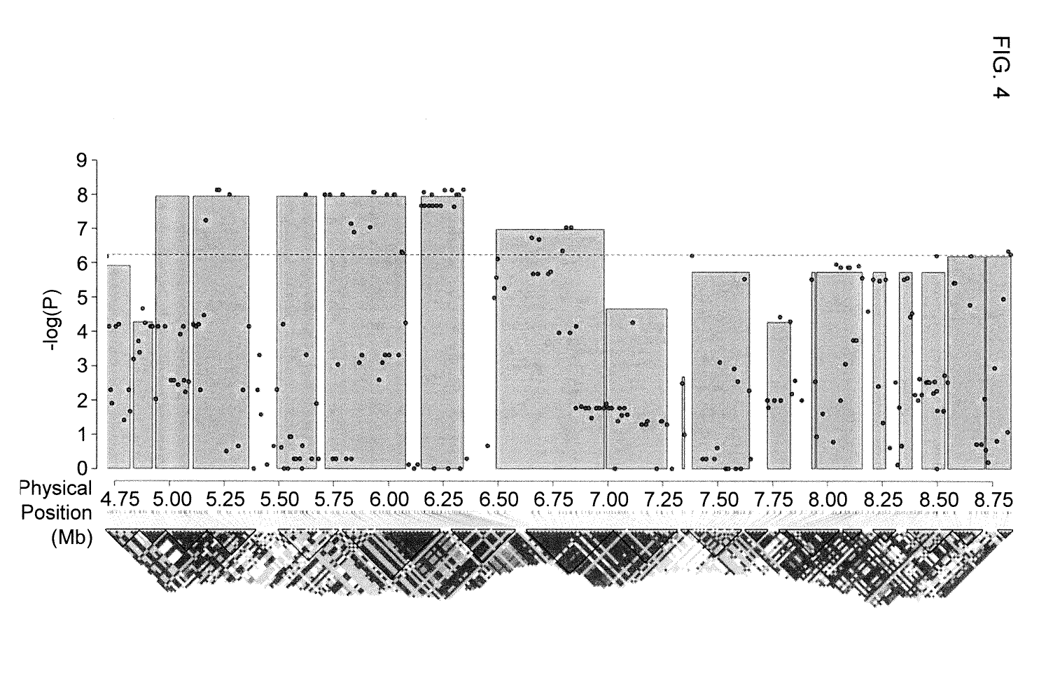

Haplotype analysis using Haploview (Barrett et al. 2005) identified 19 haplotype blocks across a 4.1 Mb region of DLA II on chromosome 12, all of which were associated with an increased risk for developing NME with P values ranging from 2.1×10⁻³ to 1.13×10⁻⁸ (FIG. 4 and Table 3). A 4.1 Mb region located from positions 4,713,392 to 8,834,652 of the genomic DNA sequence available in the canFam2 Canine genomic sequence database on Chromosome 12 (SEQ ID NO:1) is shown in FIG. 4 with 19 haplotype blocks generated in Haploview (Gabriel et al. 2002). Negative log P values from single SNP associations were derived from genome-wide analysis after removal of population outliers and individual SNPs were plotted as red circles. The horizontal dotted line represents the threshold for significant association after Bonferroni correction of −log(P)>6.24.

TABLE 3
Haplotype blocks generated by Haploview for DLA II region of
chromosome 12

| | | Population | Case | Control | Start-end | |
|---|---|---|---|---|---|---|
| Block | Haplotype | frequency | frequency | frequency | position | SEQ ID |
| 1 | AAGGAGAAA | 0.59 | 0.84 | 0.43 | 4713392- | SEQ ID NO: 3 |
| | | | | | 4821633 | |
| 2 | AGGCAAG | 0.60 | 0.80 | 0.47 | 4836721- | SEQ ID NO: 4 |
| | | | | | 4923170 | |
| 3 | GCAGGAGGGGA | 0.49 | 0.79 | 0.30 | 4938082- | SEQ ID NO: 5 |
| | | | | | 5088561 | |
| 4 | AAAACAGGAAGAG | 0.49 | 0.79 | 0.30 | 5108726- | SEQ ID NO: 6 |
| | | | | | 5364188 | |
| 5 | AGAAAGCGA | 0.49 | 0.79 | 0.30 | 5491709- | SEQ ID NO: 7 |
| | | | | | 5672682 | |
| 6 | AAGGAGAAAGAGA | 0.49 | 0.79 | 0.30 | 5710832- | SEQ ID NO: 8 |
| | GAATGGACA | | | | 6078099 | |
| 7 | GAAGAGAAGGACA | 0.49 | 0.79 | 0.30 | 6149213- | SEQ ID NO: 9 |
| | GA | | | | 6342204 | |
| 8 | GGAACAGGAAAAG | 0.51 | 0.79 | 0.33 | 6492201- | SEQ ID NO: 10 |
| | AGAGGGCGAA | | | | 6982375 | |
| 9 | CGGGAAAAGGGGA | 0.67 | 0.88 | 0.53 | 6992493- | SEQ ID NO: 11 |
| | GGC | | | | 7270218 | |
| 10 | AG | 0.73 | 0.88 | 0.64 | 7338759- | SEQ ID NO: 12 |
| | | | | | 7350261 | |
| 11 | AAAAAG | 0.50 | 0.75 | 0.34 | 7384390- | SEQ ID NO: 13 |
| | | | | | 7643147 | |
| 12 | AGGAGA | 0.70 | 0.89 | 0.58 | 7725530- | SEQ ID NO: 14 |
| | | | | | 7830733 | |
| 13 | AG | 0.50 | 0.75 | 0.34 | 7927872- | SEQ ID NO: 15 |
| | | | | | 7944953 | |
| 14 | AATAAAAGAGACGA | 0.50 | 0.75 | 0.34 | 7950821- | SEQ ID NO: 16 |
| | | | | | 8158994 | |
| 15 | GGAGG | 0.50 | 0.75 | 0.34 | 8208369- | SEQ ID NO: 17 |
| | | | | | 8265940 | |
| 16 | GCAAAG | 0.50 | 0.75 | 0.34 | 8327142- | SEQ ID NO: 18 |
| | | | | | 8386063 | |
| 17 | GGCGAGCAAAA | 0.50 | 0.75 | 0.34 | 8429601- | SEQ ID NO: 19 |
| | | | | | 8533350 | |
| 18 | ATGAACCG | 0.51 | 0.77 | 0.34 | 8546686- | SEQ ID NO: 20 |
| | | | | | 8713747 | |
| 19 | GGCA | 0.51 | 0.77 | 0.34 | 8719506- | SEQ ID NO: 21 |
| | | | | | 8834652 | |

Manually forcing all of these haplotype blocks into a single haplotype resulted in the creation of a 4.1 Mb haplotype containing 241 SNPs (SEQ ID NO: 1). This haplotype was common and strongly associated with an increased risk of developing NME at a case frequency of 85.1%, control frequency at 38.4% with significance p value at 7.97×10⁻⁷. The strong association of the NME disease with DLA II supports an autoimmune etiology. Haplotype analysis of the DLA II region identified a large, common block strongly associated with altered disease risk. Polygenic loci with MHC II polymorphisms show the strongest disease association, as observed between DLA II and NME association.

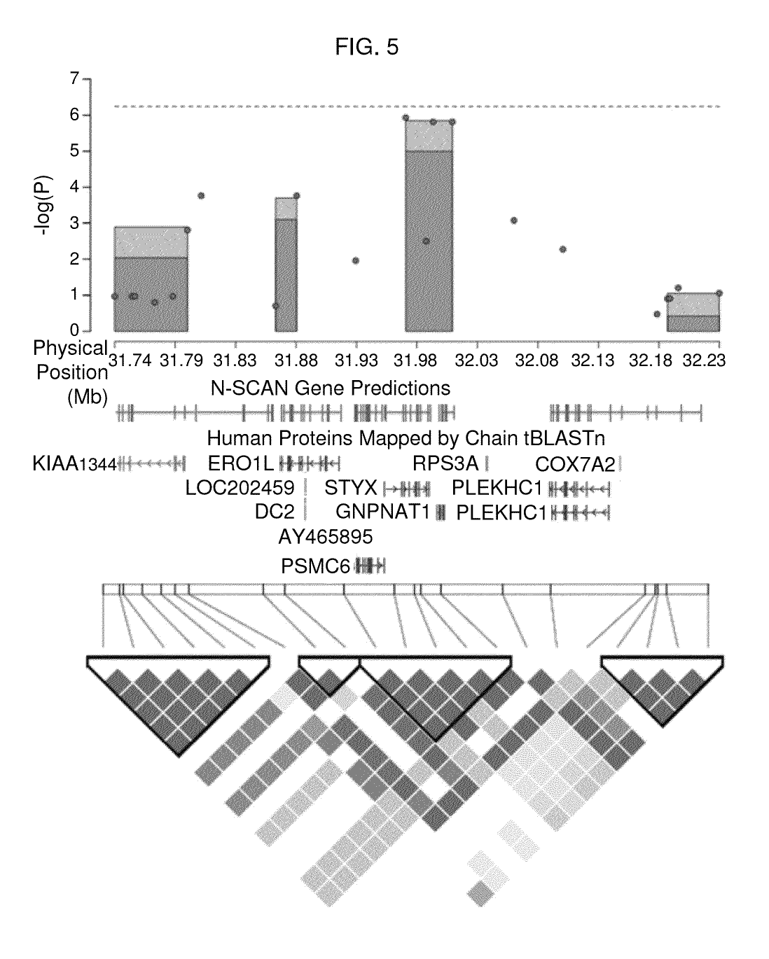

Haplotype analysis of the STYX region of chromosome 8 identified four haplotypes (FIG. 5 and Table 4). A 488 kb region located from positions 31,736,206 to 32,225,068 of the genomic DNA sequence available in the canFam2 Canine genomic sequence database (SEQ ID NO:2) is shown in FIG. 3 with 4 haplotype blocks generated in Haploview. Negative log P values from single SNP associations were derived from genome-wide analysis after removal of population outliers and individual SNPs were plotted as red circles. The horizontal dotted line represents the threshold for significant association after Bonferroni correction of −log(P)>6.24. The additional blue region within each haplotype block represents MaxT 100,000 permuted haplotypes.

TABLE 4
Haplotype blocks generated by Haploview for STYX region of chromosome 8

| | | Population | Case | Control | Start | End | |
|---|---|---|---|---|---|---|---|
| Block | Haplotype | frequency | frequency | frequency | position | Position | SEQ ID |
| 20 | GAAGGG | 0.74 | 0.59 | 0.83 | 31736206 | 31795128 | SEQ ID NO: 22 |
| 21 | AG | 0.47 | 0.27 | 0.59 | 31866373 | 31883390 | SEQ ID NO: 23 |
| 22 | GGAG | 0.52 | 0.27 | 0.68 | 31971609 | 32009283 | SEQ ID NO: 24 |
| 23 | AGAA | 0.50 | 0.59 | 0.44 | 32183184 | 32225068 | SEQ ID NO: 25 |

The most significantly associated and common haplotype spanned the STYX and GNPNAT1 genes and was protective based on phenotype (P=1.43×10⁻⁶). This block also contained two additional haplotypes significantly associated with NME risk (p˜0.005, data not shown).

STYX, (serine/threonine/tyrosine interacting protein) is a pseudophosphatase that lacks intrinsic catalytic activity and is structurally similar to members of the dual-specificity phosphatase subfamily of protein tyrosine phosphatases. Protein tyrosine phosphatases play a key role in immune system function including lymphocyte activation, with mutations in PTPN22 having been documented in association with autoimmunity (Nang et al. 2005). STYX also is known to bind to the calcineurin substrate CRHSP-24, and calcineurin plays an important role in T cell activation. Dephosphorylation of CRHSP-24 can be prevented by administration of the immunomodulatory drugs cyclosporine and FK506.

GNPNAT1, glucosamine-phosphate N-acetyltransferase 1, is involved in amino sugar metabolism including the formation of uridine diphospho-N-acetylglucosamine (UDP-GlcNAc). UDP-GlcNAc is an important cellular metabolite necessary for the synthesis of N-linked and O-linked glycans that play important roles in normal thymic T-cell apoptosis. The disruption of GNPNAT1 is expected to lead to aberrant immune responses in NME.

We claim:

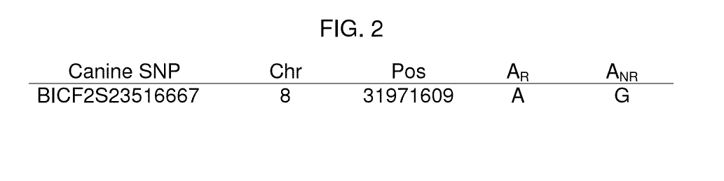

1. An isolated nucleic acid molecule selected from the group consisting of: (a) an isolated nucleic acid molecule comprising the sequence of SEQ ID NO:1; (b) an isolated nucleic acid molecule comprising the sequence of SEQ ID NO:2 (c) an isolated nucleic acid molecule comprising at least 10 nucleotides of SEQ ID NO:1, wherein said nucleic acid molecule includes at least one alternative base as listed in FIG. 1; (d) an isolated nucleic acid molecule comprising at least 10 nucleotides of SEQ ID NO:2, wherein said nucleic acid molecule includes at least one alternative base as listed in FIG. 2; and (e) an isolated nucleic molecule which is complementary to the isolated nucleic acid molecule of (a), (b), (c) and (d).

2. The isolated nucleic acid molecule of claim 1 which is a probe or primer.

3. The isolated nucleic acid molecule of claim 1, wherein said isolated nucleic acid molecule comprises two or more bases as listed in FIG. 1 and the two or more bases are in linkage disequilibrium with one another such that an allele at one of said two or more bases is predictive of an allele at the other of said two or more bases.

4. The isolated nucleic acid molecule of claim 1 comprising a SNP allele or haplotype that is associated with a risk of developing Necrotizing Meningoencephalitis in a subject.

5. The isolated nucleic acid molecule of claim 1 comprising one or more SNPs with loci in the sequence of SEQ ID NO: 1 selected from a group consisting of nucleotide variations at the indicated position: 5166878=A or G; 5217389=G or A; 5227499=G or A; 5275229=A or T; 5622709=C or A; 5710832=A or G; 5734305=A or G; 5791672=G or A; 5829667=A or G; 5843592=G or C; 5916360=A or G; 5931001=G or A; 5935549=A or G; 5992526=A or G; 6024841=T or A; 6028685=G or A; 6059850=A or G; 6064245=C or A; 6149213=G or A; 6160615=A or C; 6164202=A or G; 6184107=G or A; 6197313=A or C; 6200280=G or A; 6218850=A or G; 6238545=A or G; 6257019=G or A; 6289014=G or A; 6299459=A or G; 6311277=C or A; 6320910=A or G; 6342204=A or C; 6653816=A or G; 6686088=G or A; 6793393=A or G; 6809061=A or G; 6832252=A or G; 8822596=C or G.

6. The isolated nucleic acid molecule of claim 5, wherein the one or more SNPs that are associated with the risk of developing Necrotizing Meningoencephalitis in a subject are selected from a group consisting of nucleotide at the indicated position: 5166878=A; 5217389=G; 5227499=G; 5275229=A; 5622709=C; 5710832=A; 5734305=A; 5791672=G; 5829667=A; 5843592=G; 5916360=A; 5931001=G; 5935549=A; 5992526=A; 6024841=T; 6028685=G; 6059850=A; 6064245=C; 6149213=G; 6160615=A; 6164202=A; 6184107=G; 6197313=A; 6200280=G; 6218850=A; 6238545=A; 6257019=G; 6289014=G; 6299459=A; 6311277=C; 6320910=A; 6342204=A; 6653816=A; 6686088=G; 6793393=A; 6809061=A; 6832252=A; 8822596=C.

7. The isolated nucleic acid molecule of claim 6, wherein the subject is a canine species selected from a group consisting of Pug Dog, Chihuahua, West Highland White Terrier, Pekingese, Labrador retriever, Golden retriever, Beagle, German shepherd, Dachshund, Yorkshire terrier, Boxer, Poodle, Shih tzu, Miniature schnauzer, Pomeranian, Cocker spaniel, Rottweiler, Bulldog, Shetland sheepdog, Boston terrier, Miniature pinscher, Maltese, German shorthaired pointer, Doberman pinscher, Siberian husky, Pembroke welsh corgi, Basset hound, Bichon frise, and other existing or non-existing breeds.

8. The isolated nucleic acid molecule of claim 1 comprising a SNP with nucleotide variation at the indicated position: 31971609=A or Gin the sequence of SEQ ID NO: 2.

9. The isolated nucleic acid molecule of claim 7, wherein the SNP that is associated with the risk of developing Necrotizing Meningoencephalitis in a subject comprising the following nucleotides at the indicated position: 31971609=A;

10. The isolated nucleic acid molecule of claim 9, wherein the subject is a canine species selected from a group consisting of Pug Dog, Chihuahua, West Highland White Terrier, Pekingese, Labrador retriever, Golden retriever, Beagle, German shepherd, Dachshund, Yorkshire terrier, Boxer, Poodle, Shih tzu, Miniature schnauzer, Pomeranian, Cocker spaniel, Rottweiler, Bulldog, Shetland sheepdog, Boston terrier, Miniature pinscher, Maltese, German shorthaired pointer, Doberman pinscher, Siberian husky, Pembroke welsh corgi, Basset hound, Bichon frise, and other existing or non-existing breeds.

11. An isolated nucleic acid molecule that specifically hybridizes under stringent conditions to said isolated nucleic acid molecule of claim 1.

12. An isolated nucleic acid molecule comprising at least one nucleic acid segment of SEQ ID NO: 1, wherein said segment consisting of nucleotide positions 4,713,392 to 8,834,652 of SEQ ID NO: 1 comprises one or more SNP haplotype blocks represented by: SEQ ID NO: 3 at position 4713392-4821633, SEQ ID NO:4 at position 4836721-4923170, SEQ ID NO:5 at position 4938082-5088561, SEQ ID NO:6 at position 5108726-5364188, SEQ ID NO:7 at position 5491709-5672682, SEQ ID NO:8 at position 5710832-6078099, SEQ ID NO:9 at position 6149213-6342204, SEQ ID NO:10 at position 6492201-6982375, SEQ ID NO:11 at position 6992493-7270218, SEQ ID NO:12 at position 7338759-7350261, SEQ ID NO:13 at position 7384390-7643147, SEQ ID NO:14 at position 7725530-7830733, SEQ ID NO:15 at position 7927872-7944953, SEQ ID NO:16 at position 7950821-8158994, SEQ ID NO:17 at position 8208369-8265940, SEQ ID NO:18 at position 8327142-8386063, SEQ ID NO:19 at position 8429601-8533350, SEQ ID NO:20 at position 8546686-8713747, and SEQ ID NO:21 at position 8719506-8834652 on Chromosome 12.

13. An isolated nucleic acid molecule comprising at least one nucleic acid segment of SEQ ID NO: 2, wherein said segment consisting of nucleotide positions 31,736,206 to 32,225,068 of SEQ ID NO: 2 comprises one or more SNP haplotype blocks represented by: SEQ ID NO: 24 at position 31,736,206 to 31,795,128, SEQ ID NO: 25 at position 31,866,373 to 31,883,390, SEQ ID NO: 26 at position 31,971,609 to 32,009,283, and SEQ ID NO: 27 at position 32,183,184 to 32,225,068 on Chromosome 8.

14. A method of classifying a subject to an NME disease risk group, comprising:
receiving a nucleic acid-containing sample from the subject;
detecting the presence of at least one haplotype block listed in Table 3 and Table 4, wherein the haplotype block comprises at least two linked markers within the haplotype block, wherein the haplotype block is represented by: SEQ ID NO: 3 at position 4713392-4821633, SEQ ID NO:4 at position 4836721-4923170, SEQ ID NO:5 at position 4938082-5088561, SEQ ID NO:6 at position 5108726-5364188, SEQ ID NO:7 at position 5491709-5672682, SEQ ID NO:8 at position 5710832-6078099, SEQ ID NO:9 at position 6149213-6342204, SEQ ID NO:10 at position 6492201-6982375, SEQ ID NO:11 at position 6992493-7270218, SEQ ID NO:12 at position 7338759-7350261, SEQ ID NO:13 at position 7384390-7643147, SEQ ID NO:14 at position 7725530-7830733, SEQ ID NO:15 at position 7927872-7944953, SEQ ID NO:16 at position 7950821-8158994, SEQ ID NO:17 at position 8208369-8265940, SEQ ID NO:18 at position 8327142-8386063, SEQ ID NO:19 at position 8429601-8533350, SEQ ID NO:20 at position 8546686-8713747, and SEQ ID NO:21 at position 8719506-8834652 on Chromosome 12, and at position 32,183,184 to 32,225,068 on Chromosome 8; and
classifying the subject into a risk group based upon the presence of at least one haplotype block, whereas classifying the subject into a non-risk group based upon the absence of any haplotype block.

15. The method of claim 14 wherein detecting the haplotype block further comprises a method selected from the group consisting of Sanger sequencing, pyrosequencing, SOLID sequencing, massively parallel sequencing, barcoded DNA sequencing, PCR, real-time PCR, quantitative PCR, microarray analysis of genomic DNA, restriction fragment length polymorphism analysis, allele specific ligation, and comparative genomic hybridization.

16. The method of claim 14 wherein the marker is detected indirectly.

17. The method of claim 16 wherein detecting the marker further comprises microarray analysis of RNA, RNA in situ hybridization, RNAse protection assay, Northern blot, reverse transcription PCR, quantitative PCR, quantitative reverse transcription PCR, quantitative real-time reverse transcription PCR, reverse transcription treatment followed by direct sequencing, flow cytometry, immunohistochemistry, ELISA, Western blot, immunoaffinity chromatograpy, HPLC, mass spectrometry, protein microarray analysis, PAGE analysis, isoelectric focusing, and 2-D gel electrophoresis.

18. The method of claim 14 wherein the haplotype block is associated with a high risk of developing NME and wherein the risk group comprises subjects with a high risk of developing NME.

19. The method of claim 1 wherein the subject is a canine species selected from a group consisting of Pug Dog, Chihuahua, West Highland White Terrier, Pekingese, Labrador retriever, Golden retriever, Beagle, German shepherd, Dachshund, Yorkshire terrier, Boxer, Poodle, Shih tzu, Miniature schnauzer, Pomeranian, Cocker spaniel, Rottweiler, Bulldog, Shetland sheepdog, Boston terrier, Miniature pinscher, Maltese, German shorthaired pointer, Doberman pinscher, Siberian husky, Pembroke welsh corgi, Basset hound, Bichon frise, and other existing or non-existing breeds.

20. A set of molecular probes used in assessing the risk of developing NME of a subject comprising:
a first probe capable of detecting a first SNP selected from FIG. 1 and FIG. 2; and
a second probe capable of detecting a second SNP differing from the first SNP selected from FIG. 1 and FIG. 2.

21. The probe set of claim 20, wherein the SNPs detected by the probes are associated with a high risk of developing NME and wherein the risk group comprises subjects with a high risk of developing NME.

22. The probe set of claim 20, wherein the subject is a canine species selected from a group consisting of Pug Dog, Chihuahua, West Highland White Terrier, Pekingese, Labrador retriever, Golden retriever, Beagle, German shepherd, Dachshund, Yorkshire terrier, Boxer, Poodle, Shih tzu, Miniature schnauzer, Pomeranian, Cocker spaniel, Rottweiler, Bulldog, Shetland sheepdog, Boston terrier, Miniature pinscher, Maltese, German shorthaired pointer, Doberman pinscher, Siberian husky, Pembroke welsh corgi, Basset hound, Bichon frise, and other existing or non-existing breeds.
